(19) United States
(12) Patent Application Publication
(10) Pub. No.: US 2010/0247431 A1
(43) Pub. Date: Sep. 30, 2010

(54) POLYPEPTIDES INCLUDING MODIFIED CONSTANT REGIONS

(75) Inventors: Kathryn Lesley ARMOUR, Cambridge (GB); Michael Ronald Clark, Cambridge (GB)

Correspondence Address: NIXON & VANDERHYE, PC, 901 NORTH GLEBE ROAD, 11TH FLOOR, ARLINGTON, VA, 22203, US

(73) Assignee: Cambridge University Technical Services Limited, Cambridge (GB)

(21) Appl. No.: 12/701,729

(22) Filed: Feb. 8, 2010

Related U.S. Application Data
(63) Continuation of application No. 11/588,227, filed on Oct. 27, 2006, now abandoned.
Application No. 11/588,227 is a continuation of application No. 10/959,318, filed on Oct. 7, 2004.

(30) Foreign Application Priority Data
Oct. 17, 2003 (GB) 0324368.0

(51) Int. Cl. A61K 51/00; A61K 39/395; C07K 16/18; C07K 16/24; A61P 31/00

(52) U.S. Cl. 424/1.49; 424/130.1; 424/133.1; 424/145.1; 530/387.1; 530/387.3; 530/388.23; 530/391.3

(57) ABSTRACT

Disclosed are processes for producing a variant polypeptide (e.g. antibodies) having increased binding affinity for an FcγR, which processes comprise modifying the polypeptides by substitution of the amino acid at position 268 of a human IgG CH2 region for a non-native polar or charged amino acid e.g. Gln, Asn, Glu, or Asp. also provided are corresponding polypeptides, nucleic acids, and methods of use of the same e.g. in improved lytic therapies.

This application is a continuation of Ser. No. 11/588,227, filed Oct. 27, 2006 (published as US 2007/0041966 A1 on Feb. 22, 2007 (pending)), which is a continuation of Ser. No. 10/959,318, filed Oct. 7, 2004 (published as US 2005/0215768 A1 on Sep. 29, 2005 (pending)), which claims benefit of United Kingdom 0324368.0, filed Oct. 17, 2003, the entire contents of each of which is hereby incorporated by reference in this application.

### TECHNICAL FIELD

The present invention relates to binding polypeptides having amino acid sequences derived from a modified constant region of the immunoglobulin G (IgG) heavy chain. The invention further relates to methods and materials for producing such polypeptides, and methods and materials employing them.

### BACKGROUND ART

### Immunoglobulins

Immunoglobulins are glycoproteins which help to defend the host against infection. They generally consist of heavy and light chains, the N-terminal domains of which form a variable or V domain capable of binding antigen. The V domain is associated with constant or C-terminal domains which define the class (and sometimes subclass [isotype], and allotype [isoallotype]) of the immunoglobulin. The basic molecular structure of an antibody molecule is composed of two identical heavy chains, and two identical light chains, the chains usually being disulphide bonded together (see FIG. 10).

Thus in mammalian species immunoglobulins exist as IgD, IgG, IgA, IgM and IgE. The IgG class in turn exists as 4 subclasses in humans (IgG1, IgG2, IgG3, IgG4). There are three C-terminal domains in all of the IgG subclass heavy chains called CH1, CH2, and CH3, which are very similar between these subclasses (over 90% homology). The CH1 and CH2 domains are linked by a hinge. Structurally the fragment of an IgG antibody that consists of four of the domains from the two heavy chains, two CH2 domains and two CH3 domains, often linked by one or more disulphide bonds in the hinge region, is known as the Fc fragment, or Fc region, of the antibody. The four domains comprising of the association of the heavy and light chain V-domains together with the heavy chain CH1 and the light chain constant domains (kappa or lamda depending on light chain class), form what is known as the Fab fragment, or Fab region of the antibody (see FIG. 11). The role of the subclasses appears to vary between species.

It is known that the C-regions, and in particular the C-domains within the Fc fragment, are responsible for the various effector functions of the immunoglobulin (see Clark (1997) “IgG Effector Mechanisms” in “Antibody Engineering” Ed. Capra, Pub. Chem Immunol, Basel, Kurger, Vol 65 pp 88-110, for a detailed review).

Briefly, IgG functions are generally achieved via interaction between the Fc region of the Ig and an Fcγ receptor (FcγR) or other binding molecule, sometimes on an effector cell. This can trigger the effector cells to kill target cells to which the antibodies are bound through their variable (V) regions. Also antibodies directed against soluble antigens might form immune complexes which are targeted to FcγRs which result in the uptake (opsonisation) of the immune complexes or in the triggering of the effector cells and the release of cytokines.

In humans, three classes of FcγR have been characterised, although the situation is further complicated by the occurrence of multiple receptor forms. The three classes are:

(i) FcγRI (CD64) binds monomeric IgG with high affinity and is expressed on macrophages, monocytes, and sometimes neutrophils and eosinophils.
(ii) FcγRII (CD32) binds complexed IgG with medium to low affinity and is widely expressed. These receptors can be divided into two important types, FcγRIIa and FcγRIIb. The ‘a’ form of the receptor is found on many cells involved in killing (e.g. macrophages, monocytes, neutrophils) and seems able to activate the killing process, and occurs as two alternative alleles.

The ‘b’ form seems to play a role in inhibitory processes and is found on B-cells, macrophages and on mast cells and eosinophils. On B-cells it seems to function to suppress further immunoglobulin production and isotype switching to say for example the IgE class. On macrophages, the b form acts to inhibit phagocytosis as mediated through FcγRIIa. On eosinophils and mast cells the b form may help to suppress activation of these cells through IgE binding to its separate receptor.

(iii) FcγRIII (CD16) binds IgG with medium to low affinity and exists as two types. FcγRIIIa is found on NK cells, macrophages, eosinophils and some monocytes and T cells and mediates ADCC. FcγRIIIb is highly expressed on neutrophils. Both types have different allotypic forms.

As well as binding to FcγRs, IgG antibodies can activate complement and this can also result in cell lysis, opsonisation or in cytokine release and inflammation. The Fc region also mediates such properties as the transportation of IgGs to the neonate (via the so-called “FcRn”); increased half-life (also believed to be effected via an FcRn-type receptor—see Ghetie and Ward (1997) Immunology Today 18, 592-598) and self-aggregation. The Fc-region is also responsible for the interaction with protein A and protein G (which interaction appears to be analogous to the binding of FcRn).

### Engineering Immunoglobulins for Therapy

A common desire in the use of antibodies therapeutically is to cause cellular lysis or destruction. This is particularly true in cancer therapy where there is an obvious aim to kill the cancer cells bearing surface antigens recognised by the antibody, however other examples of lytic therapy are the use of antibody to deplete cells such as lymphocytes for example in the immunosuppression of organ graft rejection, or the prevention of graft versus host disease, or in the treatment of autoimmunity. Antibodies to antigens such as the CD52 antigen as exemplified by the CAMPATH-1 series of antibodies demonstrate by example the usefulness of this approach in a range of therapeutic disorders. The CAMPATH-1 antibody was originally developed as an IgM antibody which was very effective in lysing lymphocytes in-vitro using human serum as a complement source (Hale et al 1983). The antigen was identified as CD52 which is a small GPI-anchored glycoprotein expressed by lymphocytes and monocytes but not by haemopioetic stem cells (Xia et al 1991). It represents an exceptionally good target for complement lysis. An original therapeutic use for the IgM antibody was to remove lymphocytes from donor bone-marrow prior to engraftment to prevent graft-versus-host disease. The IgM antibody and the rat IgG2b antibody have been used regularly by a large number of bone-marrow transplantation centres world wide for this purpose (Hale and Waldmann 1996).

Although the rat IgM and also the rat IgG2a CAMPATH-1 (CD52) antibodies worked well for lysing lymphocytes in-vitro, early attempts to treat CD52 positive lymphomas/leukaemias proved unsuccessful (Dyer et al 1990). However in-vitro studies had indicated that rat IgG2b antibodies might be able to activate human FcγR mediated effector functions, in particular antibody-dependent cellular cytotoxicity (ADCC) through human FcγRIII K-cells. A rat IgG2b class-switch variant of the rat IgG2a CAMPATH-1 antibody was selected and this was tried in patients in which the IgM or IgG2a had failed to clear their CD52 tumour cells. The rat IgG2b antibody CAMPATH-1G was found to be highly efficient in clearing CD52 positive lymphocytes in-vivo indicating the importance of FcγR mediated mechanisms for in-vivo cell clearance. The CAMPATH-1G went on to be used for both lymphoma/leukaemia therapy as well as for immunosuppression in organ transplantation (Dyer et al 1990). However the major complication in the use of CAMPATH-1G was a rapid onset of a rat specific antiglobulin response in a majority of patients treated. This antiglobulin response tended to restrict the course of treatment with the antibody to one course of antibody of about 10 days duration (Dyer et al 1990). To solve the problem of the antiglobulin response the antibody was humanised by CDR grafting and a comparison of the four human subclasses IgG1, IgG2, IgG3 and IgG4 demonstrated that IgG1 was the most appropriate choice to select for an antibody which best activated human complement and bound to human Fc receptors, and which also caused cell destruction through ADCC (Riechmann et al 1988). The humanised antibody expressed as a human IgG1 turned out to be effective in depleting leukaemic cells and inducing remission in patients (Hale et al 1988, Dyer et al 1990).

Following the successful use of the humanised antibody CAMPATH-1H in lymphoma/leukaemia therapy the antibody was used in a number of other disorders where immunosuppression was the desired outcome. CAMPATH-1H has been used in the treatment of patients with a number of diseases with autoimmune involvement including refractory rheumatoid arthritis as well as patients with systemic vasculitis and also multiple sclerosis (Lockwood et al 1993, Maithieson et al 1990, Matteson et al 1995, Moreau et al 1994). In each case efficacy of a lytic antibody has been demonstrated.

In the engineering of a recombinant version of the humanised antibody Campath-1H (Riechmann et al 1988) a number of different antibodies with different human IgG constant regions were compared for their abilities to interact with complement and with Fc receptors and to kill cells using CDC or ADCC. These studies and other similar studies revealed that the IgG1 isotype proved to be superior to other IgG subclasses and was the subclass of choice for human therapy where lysis of cells was the main goal. Clinical trials with Campath-1H as an IgG1 proved successful and so the antibody finally achieved FDA approval in for lymphocytic leukeamia therapy under the trademark name CAMPATH® (Trademark of Ilex-Oncology Inc).

Mutant constant regions are also discussed by Armour et al (2003) “Differential binding to human FcγRIIa and FcγRIIb receptors by human IgG wildtype and mutant antibodies” Mol Immunol. 2003 December; 40(9):585-93.

WO00/42072 concerns polypeptides comprising a variant Fc region, and in particular Fc region-containing polypeptides that have altered effector functions as a consequence of one or more amino acid modifications in the Fc region thereof.

It can be seen from the forgoing that the provision of methods or materials for modifying effector functions, for example by engineering of IgG Fc regions to improve their receptor binding properties, would provide a contribution to the art.

### DISCLOSURE OF THE INVENTION

The present inventors have used novel modifications of Fc regions (in particular human IgG CH2 regions) to alter their effector function, and in particular to increase the binding levels or signaling ability of polypeptides comprising those regions to Fcγ receptors (FcγRs).

The manner by which the sequences were developed, and certain demonstrated properties, will be discussed in more detail hereinafter. However, briefly, the inventors have shown that modifying the residue at position 268 in a human IgG CH2 region, for example from H (His) to another polar amino acid such as Q (Gln) or a charged one such as E (Glu) can enhance the FcγR binding of the region. This is particularly surprising since His is native to IgG1, which is known to bind more tightly to FcγRs than IgG4 (in which Gln is native).

IgG1 antibodies including a point modification at position 268 have been prepared in the past. Shields et al. (2001, J. Biol. Chem: 276, 9: 6591-6604) appeared to show that that the modification of His 268 to neutral Ala in IgG1 had no statistically significant effect on its binding to FcγRI. Its effects on FcγRIIa and IIb were broadly equivalent to each other.

Thus in a first aspect of the present invention there is disclosed a process for increasing the binding affinity for an Fcγ receptor (FcγR) of a polypeptide,

or a process for producing a variant polypeptide having increased binding affinity for an FcγR,
which process comprises modifying a polypeptide which comprises a human IgG CH2 region by substitution of the amino acid at position 268 for a different polar or charged amino acid.

In this and all other aspects of the present invention, the numbering of the residues in the IgG Fc region is that of the EU index as in Kabat (see Kabat et al. “Sequences of proteins of immunological interest”. Bethesda, US Department of Health and Human Services, NIH, 1991):

Variant polypeptides of the present invention may be used, inter alia, in binding molecules where a higher affinity binding to an FcγR is required.

Variant polypeptides of the present invention may also be used to increase other effector functions e.g. to improve cytotoxicity (e.g. as measured by ADCC, chemiluminsescence or apoptosis).

### Fcγ Receptor

This may be any FcγR (e.g. FcγRI, FcγRII, FcγRIII, or subtypes thereof e.g. FcγRIIa or IIb, FcγRIIIa or IIIb). Preferably the mutation increases the affinity for any 2 or more of FcγRI, FcγRIIa, FcγRIIb, FcγRIIIa or FcγRIIIb, more preferably any 2 or more of FcγRI, FcγRIIa and FcγRIIb. The effects achieved with a variety of different receptors are illustrated in the Figures.

Thus the method provides for introducing one of a defined class of amino acids at position 268 into a “parent” polypeptide, which amino acid is non-native to that parent, to produce a variant thereof having increasing binding affinity to an FcγR compared with the parent.

As demonstrated in the results hereinafter, in one aspect the present invention discloses a process for increasing the relative binding affinity for one FcγRII subtype over the other subtype, of a polypeptide,

or a process for producing a variant polypeptide having that property,
which process comprises modifying a polypeptide which comprises a human IgG CH2 region by substitution of the amino acid at position 268 for a different polar or charged amino acid.

In one aspect of the invention the relative binding affinity for an FcγRIIb receptor compared to an FcγRIIa receptor may be increased. In another embodiment the relative binding affinity for an FcγRIIa receptor compared to an FcγRIIb receptor may be increased.

As discussed below, in preferred embodiments the variant polypeptides of the present invention having enhanced binding to FcγRIIb e.g. compared to wild-type IgG1 (or an improved ratio of binding of FcγRIIb to FcγRIIa e.g. compared to wild-type IgG1) may be used in general in preventing immunization to chosen antigens through co-ligation of the inhibitory receptor e.g. in suppressing a B-cell response. Additionally or alternatively such antibodies may have improved lytic or other cell killing properties e.g. owing to an improved ability to trigger apoptosis.

### Assessment of Binding Affinity

Generally the increase in affinity which the variant has for the receptor (as compared with the polypeptide which lacks the modified amino acid at position 268 from which it is derived) may, in preferred embodiments, be at least 1.5, 2, 3, 4, 5, or 10 fold, or more).

Binding affinity can be measured by any method known in the art, as appropriate to the FcγR in question (see e.g. WO99/58572 (Cambridge University Technical Services), and Examples below.

### Choice of Parent CH2 Sequence

The variant may be derived from any human IgG. Preferably the variant is derived from a human IgG1, IgG2 or IgG3 CH2 region, most preferably from IgG1 or IgG3, most preferably from IgG1.

As can be seen from FIG. 9, a significant number of monoclonal antibodies currently in clinical trials are of the IgG1 type. Examples of FDA approved antibodies which have been specifically engineered as an IgG1 for their cytoxicity include the antibodies Herceptin (Genentech, FDA approval 1998) for the treatment of breast cancer, and Retuxan (Genentech) for the treatment of B-cell lymphoma. (see also the following internet site: path.cam.ac.uk/˜mrc7/humanisation/antibodies.html). For a list of other recombinant antibodies in human therapy see reviews by Glennie & Johnson 2000 and Glennie & van de Winkel 2003. It is notable that many of these have been deliberately engineered with the human IgG1 isotype because of its greater activity in binding to human FcγR, thus inducing apoptosis and also triggering complement and cell-mediated cyctotoxicity.

The present invention provides (inter alia) a novel means of manipulating the binding of IgG1 to FcγRs (e.g. FcγRIIb) thereby manipulating and improving its one or more of its effector properties compared to wild-type IgG1. Embodiments of the present invention can demonstrate improved cell killing properties, such as apoptosis and other FcγR-mediated functions.

Preferably the modified or variant (the terms are used interchangeably) CH2 produced in the invention is derived from a native CH2 region. However it should be noted that the CH2 region need not be native, but may correspond to (be derived from) a native CH2 region, but include further amino acids deletions, substitutions or additions thereto (over and above that at position 268).

Preferably the variant CH2 region is at least 90, 91, 92, 93, 94, 95, 96, 97, 98, or 99% identical to the native CH2 region from which it, and the parent polypeptide, were derived. Identity may be assessed using the standard program BestFit with default parameters, which is part of the Wisconsin Package, Version 8, September 1994, (Genetics Computer Group, 575 Science Drive, Madison, Wis., USA, Wis. 53711). The native human IgG1, G2, G3 and G4 CH2 region sequences, from positions 231-340, are shown in FIG. 1).

Thus the variant CH2 region may include, in addition to the substitution at position 268, no more than 1, 2, 3, 4, 5, 6, 7, 8, 9 changes compared with the native CH2 region.

### Preferred Substitutions

As can be seen from FIG. 1, position 268 in IgG1, 2 and 3 is H (His).

In one embodiment of the present invention this is modified to a different polar amino acid such as Q (Gln) or N (Asn). Gln may be preferred as this may be less immunogenic, being derived from IgG4.

In another embodiment of the invention this is modified to a negatively charged amino acid such as E (Glu) or D (Asp).

These embodiments may be preferred where it is desired increase the relative binding affinity of the polypeptide for an FcγRIIb receptor compared to an FcγRIIa receptor. Conversely, where it is desired to increase the relative binding affinity of the polypeptide for an FcγRIIa receptor compared to an FcγRIIb receptor, positively charged amino acids such as K (Lys) or R (Arg) may be preferred.

The most preferred C_{H}2 sequences are shown in FIG. 2, as aligned with IgG1. Most preferred sequences are designated G1Δd and G1Δe.

As discussed above, other preferred CH2 regions may include no more than 1, 2, 3, 4, 5, 6, 7, 8, 9 changes with respect to any C_{H}2 sequences are shown in FIG. 2 (but wherein position 268 is unchanged compared to those C_{H}2 sequences). Optional other changes include those described WO99/58572 (Cambridge University Technical Services).

Preferably, where the identity of the residue at position 268 is a Gln, and the variant derives from IgG1, residue 274 will be native to IgG1 i.e. lys.

Preferably, where the identity of the residue at position 268 is a Gln, and the variant derives from IgG2, residue 309 should be native to IgG2 i.e. Val.

Preferably, where the identity of the residue at position 268 is a Gln, and the variant derives from IgG3, residue 276 should be native to IgG3 i.e. lys.

Changes to the depicted sequences which to conform with known human allotypic variation are also specifically embraced by the present invention—for example where the variant derives from IgG2, residue 282 may optionally be Met, which is an alternative allotype.

In all cases, it is preferred that the identity of the residue at position 297 is a Asn, and that this is glycosylated in the polypeptide.

### Polypeptides

The variant polypeptide may consist, or consist essentially of, the CH2 sequences discussed above. However, preferably, the variant polypeptide comprises an entire constant region of a human IgG heavy chain, comprising the CH2 above.

Thus any of the CH2 sequences discussed herein may be combined with (e.g. run contiguously with) natural or modified C_{H}3 and natural or modified hinge region, plus optionally C_{H}1, sequences in the molecules of the present invention. Thus, for example, a variant polypeptide based on the human IgG1 CH2 region may be present with the IgG1 CH1 and CH3 regions.

Numerous sequences for human C regions have been published; see e.g. Clark (1997) supra. Other sequences for human immunoglobulin heavy chains can be obtained from the SwissProt and PIR databases using Lasergene software (DNAStar Limited, London UK) under accession numbers A93433, B90563, A90564, B91668, A91723 and A02146 for human Igγ-1 chain C region, A93906, A92809, A90752, A93132, A02148 for human Igγ-2 chain C region, A90933, A90249, A02150 for human Igγ-4 chain C region, and A23511 for human Igγ-3 chain C region.

Thus in one aspect the present invention provides a variant polypeptide, which may be one which is obtained or obtainable by the process described above

Thus this aspect provides a variant polypeptide having increased binding affinity to an Fcγ receptor (FcγR), which polypeptide comprises a human IgG CH2 region in which the amino acid at position 268 has been substituted for a different polar or charged amino acid, preferably negatively charged amino acid.

As described above, the variant polypeptide may have increased relative binding affinity for one of the FcγRII subtypes over the other. The amino acid at position 268 of the variant polypeptide will be a different polar or charged amino acid to that found in the corresponding native CH2 region. Preferably the variant is derived from a human IgG1, IgG2 or IgG3 CH2 region, most preferably from IgG1. Preferably the amino acid at position 268 of the variant polypeptide is Q (Gln), N (Asn), E (Glu) or D (Asp).

### Binding Molecules

Preferably the polypeptide is a binding molecule comprising:

(i) a binding domain capable of binding a target molecule, and
(ii) an effector domain comprising an a variant CH2 polypeptide as described above, and more preferably comprising an entire IgG constant region of the invention.

Preferred target molecules and corresponding binding domains, and also uses of such binding molecules, are discussed in more detail hereinafter.

Thus, although the effector domain will generally derive from an antibody, the binding domain may derive from any molecule with specificity for another molecule e.g. an enzyme, a hormone, a receptor (cell-bound or circulating) a cytokine or an antigen (which specifically binds an antibody). As used herein, the term “immunoadhesin” designates antibody-like molecules which combine such binding domains with an immunoglobulin constant domain.

Preferably, it comprises all or part of an antibody or a derivative thereof, particularly a natural or modified variable domain of an antibody. Thus a binding molecule according to the present invention may provide a rodent or camelidae (see WO 94/25591) originating antibody binding domain and a human immunoglobulin heavy chain as discussed above. More preferably the binding molecule is a humanised antibody.

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. Thus the term includes molecules having more than one type of binding domain, such as bispecific antibodies (see e.g. PCT/US92/09965). In these cases one ‘arm’ binds to a target cell and the other binds to a second cell to trigger killing of the target. In such cases it may be desirable to minimise the impact the effector portion, which might otherwise activate further cells which interfere with the desired outcome. The ‘arms’ themselves (i.e. the binding domain) may be based on Ig domains (e.g. Fab) or be from other proteins as in a fusion protein, as discussed in more detail below.

The binding molecule may comprise more than one polypeptide chain in association e.g. covalent or otherwise (e.g. hydrophobic interaction, ionic interaction, or linked via sulphide bridges). For instance it may comprise a light chain in conjunction with a heavy chain comprises the effector domain. Any appropriate light chain may be used e.g. the most common kappa light chain allotype is Km(3) in the general population. Therefore it may be desirable to utilise this common kappa light chain allotype, as relatively few members of the population would see it as foreign.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity (see e.g. Jones et al., Nature 321:522-525 (1986); Riechmann et al., Nature 332:323-329 (1988); and Presta, Curr. Op. Struct. Biol. 2:593-596 (1992)).

Methods of producing antibodies (and hence binding domains) include immunising a mammal (e.g. human, mouse, rat, rabbit, horse, goat, sheep, camel or monkey) with a suitable target protein or a fragment thereof. Antibodies may be obtained from immunised animals using any of a variety of techniques known in the art, and might be screened, preferably using binding of antibody to antigen of interest. For instance, Western blotting techniques or immunoprecipitation may be used (Armitage et al, 1992, Nature 357: 8082). Cloning and expression of Chimaeric antibodies is described in EP-A-0120694 and EP-A-0125023.

However it will be appreciated by those skilled in the art that there is no requirement that other portions of the polypeptide (or other domains of the molecule) comprise natural sequences—in particular it may be desirable to combine the sequence modifications disclosed herein with others, for instance selected from the literature, provided only that the required activities are retained. The skilled person will appreciate that binding molecules comprising such additionally-modified (e.g. by way of amino acid addition, insertion, deletion or substitution) effector domains fall within the scope of the present invention. For example certain ‘null allotype’ sequences are disclosed in WO 92/16562.

The binding and effector domains may be combined by any suitable method. For instance domains may be linked covalently through side chains. Alternatively, sulphydryl groups generated by the chemical reduction of cysteine residues have been used to cross-link antibody domains (Rhind, S K (1990) EP 0385601 Cross-linked antibodies and processes for their preparation). Finally, chemical modification of carbohydrate groups has been used to generate reactive groups for cross-linking purposes. These methods are standard techniques available to those skilled in the art. They may be particularly applicable in embodiments wherein the binding polypeptide contains non-protein portions or groups.

Generally it may be more appropriate to use recombinant techniques to express the binding molecule in the form of a fusion protein. Methods and materials employing this approach form further aspects of the present invention, as set out below.

### Nucleic Acids

Preferably the processes described hereinbefore are performed by recombinant DNA technology e.g. site-directed mutagenesis or by via PCR using mutagenic primers. For example, nucleic acid encoding the CH2 domain can be generated, in the light of the present disclosure, by site directed mutagenesis, for instance by methods disclosed herein or in the published art (see e.g. WO 92/16562 or WO 95/05468 both of Lynxvale Ltd; also Kunkel et al., Proc. Natl. Acad. Sci. USA 82:488 (1987)).

Thus a process according to the present invention may comprise:

(i) providing a nucleic acid comprising a polynucleotide sequence encoding a human IgG CH2 region,
(ii) modifying the codon corresponding to amino acid at position 268 such that it encodes a different polar or charged (preferably negatively charged) amino acid,
(iii) causing or allowing expressing of said modified polynucleotide sequence (e.g. as present in a vector or other construct, as described below) in a suitable host cell, such as to produce a variant polypeptide having increased binding affinity to an FcγR.

The variant polypeptide may have increased relative binding affinity for one of the FcγRII subtypes over the other.

The polynucleotide sequence may encode an entire constant region of a human IgG heavy chain and optionally a binding domain capable of binding a target molecule.

Alternatively following step (ii) the modified polynucleotide sequence may be recombined with other polynucleotide sequences e.g. encoding other constant regions of a human IgG heavy chain and\or a binding domain capable of binding a target molecule.

### Nucleic Acid Products

In another aspect the present invention provides a modified nucleic acid obtained or obtainable by the process described above

Thus this aspect provides a nucleic acid comprising a polynucleotide sequence encoding a variant polypeptide having increased binding affinity to an FcγR, which polypeptide comprises a human IgG CH2 region in which the amino acid at position 268 has been substituted for a different polar or (preferably negatively) charged amino acid

Preferably the modified polynucleotide is derived from a human IgG1, IgG2 or IgG3 CH2 sequence, most preferably from IgG1.

Thus the codon corresponding to amino acid at position 268 in the polynucleotide encodes a different polar or charged amino acid to that found in the corresponding native CH2 region. Preferably it will encode Q (Gln), N (Asn), E (Glu) or D (Asp).

Nucleic acid according to the present invention may include cDNA, RNA, genomic DNA (including introns) and modified nucleic. Where a DNA sequence is specified, e.g. with reference to a Figure, unless context requires otherwise the RNA equivalent, with U substituted for T where it occurs, is encompassed.

Nucleic acid molecules according to the present invention may be provided isolated and/or purified from their natural environment, in substantially pure or homogeneous form, or free or substantially free of other nucleic acids of the species of origin. Where used herein, the term “isolated” encompasses all of these possibilities.

The nucleic acid molecules will be wholly or partially synthetic—in particular they will be recombinant in that nucleic acid sequences (or substitutions) which are not found together in nature have been ligated or otherwise combined artificially.

In a further aspect there is disclosed a nucleic construct, e.g. a replicable vector, comprising the nucleic acid sequence.

A vector including nucleic acid according to the present invention need not include a promoter or other regulatory sequence, particularly if the vector is to be used to introduce the nucleic acid into cells for recombination into the genome.

Preferably the nucleic acid in the vector is under the control of, and operably linked to, an appropriate promoter or other regulatory elements for transcription in a host cell such as a microbial, (e.g. bacterial, yeast, filamentous fungal) or eucaryotic (e.g. insect, plant, mammalian) cell.

Particularly, the vector may contain a gene (e.g. gpt) to allow selection in a host or of a host cell, and one or more enhancers appropriate to the host.

The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell.

By “promoter” is meant a sequence of nucleotides from which transcription may be initiated of DNA operably linked downstream (i.e. in the 3′ direction on the sense strand of double-stranded DNA). The promoter may optionally be an inducible promoter.

“Operably linked” means joined as part of the same nucleic acid molecule, suitably positioned and oriented for transcription to be initiated from the promoter.

Thus this aspect of the invention provides a gene construct, preferably a replicable vector, comprising a promoter operatively linked to a nucleotide sequence provided by the present invention.

Generally speaking, those skilled in the art are well able to construct vectors and design protocols for recombinant gene expression. Suitable vectors can be chosen or constructed, containing appropriate regulatory sequences, including promoter sequences, terminator fragments, polyadenylation sequences, enhancer sequences, marker genes and other sequences as appropriate. For further details see, for example, “Molecular Cloning: a Laboratory Manual: 2nd edition”, Sambrook et al, 1989, Cold Spring Harbor Laboratory Press.

Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1992. The disclosures of Sambrook et al. and Ausubel et al. are incorporated herein by reference.

Also embraced by the present invention are cells transformed by expression vectors defined above. Also provided are cell cultures (preferably rodent) and products of cell cultures containing the binding molecules.

### Binding Domains and Target Molecules

The binding molecules of the present invention comprise a binding domain capable of binding a target molecule.

The binding domain will have an ability to interact with a target molecule which will preferably be another polypeptide, but may be any target (e.g. carbohydrate, lipid (such as phospholipid) or nucleic acid). Preferably the interaction will be specific. The binding domain may derive from the same source or a different source to the effector domain.

Typically the target will be antigen present on a cell, or a receptor with a soluble ligand. This may be selected as being a therapeutic target, whereby it is desired to bind it with a molecule having the properties discussed above.

As discussed above, the target may be present on or in a target cell, for example a target cell which it is desired to lyse, or in which it is desired to induce apoptosis. Lytic therapies may be used in tumour therapies e.g. where the target is a cancer-associated antigen, whereby the combined ADCC, CDC and apoptosis induce cancer cell therapy. Other targets may be those associated with infectious diseases, or associated with diseases caused by unwanted cellular proliferation, aggregation or other build up.

Variant polypeptides (e.g. antibodies) may be used by those skilled in the art analogously to those already in use for any of these purposes (see e.g. FIG. 9, or discussion by Glennie & Johnson 2000 and Glennie & van de Winkel 2003).

In one preferred embodiment, variant polypeptides such as antibodies according to the present invention may be used in the treatment of Haemolytic Disease of the Newborn using anti-D antibodies. Anti-D prophylaxis is a successful example of the clinical application of antibody-mediated immune suppression. Passive IgG anti-D is given to Rh D-negative women to prevent immunisation to foetal Rh D-positive red blood cells (RBC) and subsequent haemolytic disease of the newborn. Antibodies of the human IgG1 and of the human IgG3 class which are known to bind to human FcγRs are injected into women who have recently been exposed to RhD red cells from their infants as a result of pregnancy. The antibodies bind to the RhD positive red blood cells and help to remove them from the mothers circulation via interactions with FcγR bearing cells. However observations made during such treatments suggest that most Rh D antigen sites on RBC are not bound by passive anti-D, and thus epitope masking (which may occur in experimental murine models using xenogeneic RBC) is not the reason why anti-D responses are prevented by administration of prophylactic anti-D.

It is thought that although clearance and destruction of the antigenic RBC may be a contributing factor in preventing immunisation, the down-regulation of antigen-specific B cells through co-ligation of B cell receptors and inhibitory IgG Fc receptors (FcγRIIb) must also occur (Reviewed by Kumpell B M 2002). Thus antibodies with enhanced binding to FcγRIIb (or an improved ratio of binding of FcγRIIb to FcγRIIa) may be used in this and other contexts where it is desired to prevent immunization to selected antigens, through co-ligation of the inhibitory receptor i.e. where it is desired to suppress a B-cell mediated immune response. Preferred indications include use in preventing allo-immunisation as in Haemolytic Disease of the Newborn (HDN) or Feto-alloimmune thrombocytopenia (FAIT), and more generally the prevention of immune reponses to allergens in the treatment of allergy and asthma.

Thus in one aspect, the invention provides a method of treating a mammal suffering from a disorder comprising administering to the mammal a therapeutically effective amount of a variant polypeptide as discussed herein.

Also provided is use of the binding molecules of the present invention to bind to a target molecule, such as those discussed above.

The present invention also provides a reagent which comprises a binding molecule as above, whether produced recombinantly or otherwise.

The present invention also provides a pharmaceutical preparation which comprises a binding molecule as above, plus a pharmaceutically acceptable carrier or diluent. The composition for potential therapeutic use is sterile and may be lyophilised.

The present invention also provides a method of treating a patient which comprises administering a pharmaceutical preparation as above to the patient, or to a sample (e.g. a blood sample) removed from that patient, which is subsequently returned to the patient.

The present invention also provides a method of treating a patient which comprises causing or allowing the expression of a nucleic acid encoding a binding molecule as described above, whereby the binding molecule exerts its effects in vivo in the patient.

Also provided is the use of a binding molecule as above in the preparation of a pharmaceutical, particularly a pharmaceutical for the treatment of the diseases discussed above e.g. by the various mechanisms discussed (which include lysis of a target cell by ADCC, CDC, or apoptosis and\or suppression of B-cell induced immune response).

The disclosure of all references cited herein, inasmuch as it may be used by those skilled in the art to carry out the invention, is hereby specifically incorporated herein by cross-reference.

The invention will now be further described with reference to the following non-limiting Figures and Examples. Other embodiments of the invention will occur to those skilled in the art in the light of these.

### BRIEF DESCRIPTION OF THE DRAWINGS

FIG. 1: shows a line up of wild-type C_{H}2 sequences from IgG1 to 4 (lgG1—SEQ ID NO:1; lgG2—SEQ ID NO:2, lgG3—SEQ ID NO:3; lgG4—SEQ ID NO:4).

FIG. 2: shows example variant C_{H}2 sequences according to the present invention, including G1Δd and G1Δacd, containing Q268, and G1Δe and G1Δace, containing E268. Some of the properties of the variants of the invention are described by FIGS. 3-8 (IgG1—SEQ ID NO:1; G1Δd—SEQ ID NO:5; G1Δe—SEQ ID NO:6; G1Δad—SEQ ID NO:7; G1Δae—SEQ ID NO:8; G1Δacd—SEQ ID NO:9; G1Δace—SEQ ID NO:10; G1Δabd—SEQ ID NO:11; G1Δabe—SEQ ID NO:12; G1Δcd—SEQ ID NO:13; G1Δce—SEQ ID NO:14; G1Δbd—SEQ ID NO:15; G1Δbe—SEQ ID NO:16; G2Δd—SEQ ID NO:17; G2Δe—SEQ ID NO:18; G3Δd—SEQ ID NO:19; G3Δe—SEQ ID NO:20; G4Δe—SEQ ID NO:21; G1Δ268N—SEQ ID NO:22; G1Δ268D—SEQ ID NO:23; G1Δ268K—SEQ ID NO:24; G1Δ268R—SEQ ID NO:25).

FIG. 3. Binding of complexes of Fog-1 antibodies to FcγRIIb-bearing cells. Fog-1 antibodies G1, G1Δd, G1Δe, G1Δac, G1Δacd and G1Δace and human IgA1, κ were pre-complexed using goat anti-human κ-chain F(ab′)₂ molecules. 3T6+FcγRIIb1* cells were incubated with these complexes and, subsequently, with FITC-conjugated rabbit F(ab′)₂ molecules specific for F(ab′)₂ fragments of goat IgG. The geometric mean of fluorescence was plotted against the concentration of test antibody. This result is typical of three independent experiments performed. G1Δd and G1Δe show a greater level of binding than IgG1, amounting to an approximate eight-fold difference in the case of G1Δe. G1Δac and G1Δacd show a similar level of binding to the IgA negative control with G1Δace binding slightly more at the top antibody concentrations.

FIG. 4. Binding of complexes of Fog-1 antibodies to FcγRIIa-bearing cells. The assay was carried out as in FIG. 3 but using 3T6+FcγRIIa 131H cells. The graph shows a typical result from three separate experiments. G1Δd shows a similar level of binding to IgG1 for this receptor whereas the binding of G1Δe is about two-fold higher. The binding curves for G1Δac, G1Δacd and G1Δace are slightly above that of the IgA negative control.

FIG. 5. Binding of Fog-1 antibodies to FcγRI-bearing cells. B2KA cells were incubated with Fog-1 antibodies, followed by biotinylated goat anti-human κ-chain antibodies and then ExtrAvidin-FITC. The geometric mean of fluorescence was plotted against the concentration of test antibody. This result is typical of three independent experiments performed. G1, G1Δd and G1Δe show a similar high level of binding. G1Δac and G1Δacd show low levels of binding at the top antibody concentrations. However, the addition of the Δe mutation to G1Δac, to give the G1Δace antibody, significantly increases binding.

FIGS. 6A and 6B. Binding of complexes of Fog-1 antibodies to FcγRIIIb-bearing cells. The assay was carried out as in FIG. 3 but using CHO cells expressing FcγRIIIb of the NA1 (part a) or NA2 (part b) allotypes. Each graph shows a typical result from three separate experiments. For both of these receptors, G1Δe shows higher binding than G1 whereas G1Δd shows slightly lower binding. G1Δac, G1Δacd and G1Δace bind weakly.

FIGS. 7A, 7B and 7C. Monocyte chemiluminescence in response to red blood cells sensitised with Fog-1 antibodies. RhD-positive RBC (O R₁R₂) were coated with the Fog-1 antibodies at the concentrations indicated and then washed. Peripheral blood mononuclear cells were isolated from blood pooled from six random donors. These were incubated with the sensitised RBC in the presence of luminal which generates light upon reaction with by-products of RBC phagocytosis. For each sample, the integral of chemiluminescence measurements taken over one hour was corrected for the value obtained for uncoated RBC. Results were expressed as a percentage of the value achieved with 4 μg/ml of a control antibody, representing maximum activation. On each of these graphs, two of the test antibodies are compared to a previously-validated Fog-1 IgG1 standard. Symbols represent duplicate results for a given antibody concentration, with a line drawn to show the mean values. It is seen that test antibodies G1 and G1Δd have the same activity as the standard whereas G1Δe is two-fold more active. G1Δac and G1Δacd have little activity but G1Δace does promote low levels of activation when cells are sensitised at concentrations above 1 μg/ml.

FIG. 8. Antibody-dependent cell-mediated cytotoxicity against RhD-positive RBC in presence of Fog-1 antibodies. Antibody samples, non-adhering peripheral blood mononuclear cells and ⁵¹Cr-labelled RBC were incubated for 16 h and then the cells pelleted. Counts of ⁵¹Cr released into the supernatant were adjusted for spontaneous lysis in the absence of antibody. For each sample, the specific lysis was expressed as a percentage of the maximum lysis (achieved with detergent). Results are shown as the mean (+/−SD) for triplicate samples. At low concentrations, two-fold less G1Δe than G1 is needed to achieve the same level of lysis. G1Δac and G1Δacd do not promote lysis although G1Δace is active at high concentrations.

FIGS. 9-1 through 9-4: This shows a selection of monoclonal antibodies in clinical development, including listing what type of antibody they are based upon (from archive.bmn.com/supp/ddt/glennie.pdf).

FIG. 10. Shown schematically is the basic IgG immunogloblin structure of two heavy (H) chains in black and two light (L) chains in white. The two heavy chains are disulphide bonded together and each light chain is disulphide bonded to a heavy chain. The antibody also has two antigen binding Fab regions and a single Fc region.

FIG. 11. This shows an alternative schematic of an IgG whereby each globular domain of the molecule is illustrated as a ellipse. The heavy chain domains are shown in darker shades and the light chain domains in lighter shades. The heavy and light chain variable domains VH and VL are also indicated along with the position of the antigen binding site at the extreme of each Fab. Each CH2 domain is glycosylated at a conserved asparagine residue number 297 and the carbohydrate sits in the space between the two heavy chains. Disulphide bridges between the chains are indicated as black dots within the flexible hinge region and between the heavy and light chains.

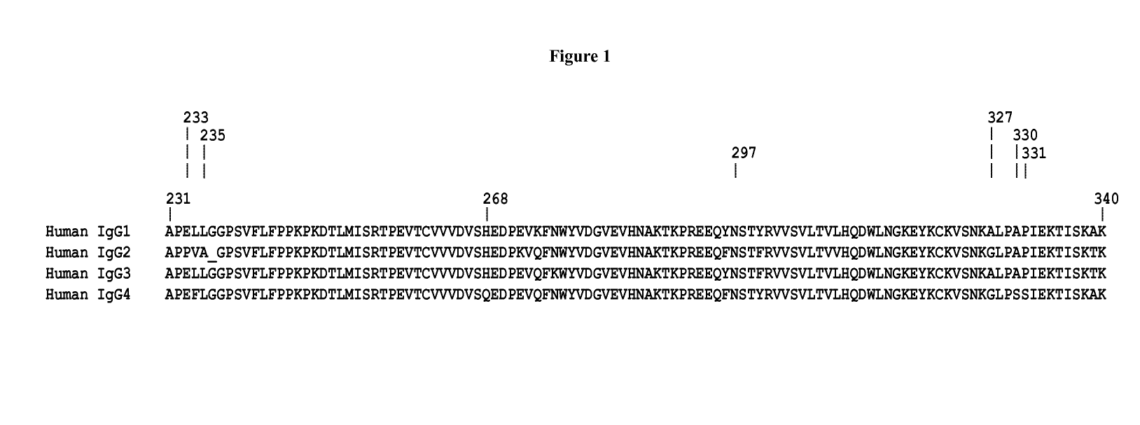

### MATERIALS AND METHODS

### Production of Antibodies

The construction of expression vectors for the wildtype IgG1, IgG2 and IgG4 genes and variants thereof (G1Δa, G1Δb, G1Δc, G1Δab, G1Δacd, G1Δace, G2Δa, G4Δb, G4Δc), their use in the production of antibodies and the testing of the effector functions of these antibodies is described in WO99/58572 (Cambridge University Technical Services), the disclosure of which is hereby incorporated by reference. Further information on the effector activities of these antibodies is also found in Armour et al (1999).

The vectors described in WO99/58572 (Cambridge University Technical Services) were used as the starting point for the construction of the heavy chain expression vectors for the Fog-1 G1Δd and Fog-1 G1Δe antibodies. As desccribed therein, the starting point for the IgG1 constant region was the human IgG1 constant region gene of allotype G1m(1,17) in a version of the vector M13tg131 which contains a modified polylinker (Clark, M. R.: WO 92/16562). The 2.3 kb IgG1 insert thus has a BamHI site at the 5′ end and contains a HindIII site adjacent to the BamHI site. At the 3′ end, downstream of the polyadenylation signal, the following sites occur in the order 5′ to 3′: SphI, NotI, BglII, BamHI.

The first procedure was to introduce an XbaI restriction site between the CH1 and hinge exons, a XhoI site between the hinge and CH2 exons and a KpnI site between the CH2 and CH3 exons in order to facilitate exchange of mutant exon sequences. This was similar to the manipulation of IgG1 and IgG4 genes carried out previously (Greenwood, J., Clark, M. and Waldmann, H. (1993) Structural motifs involved in human IgG antibody effector functions. Eur. J. Immunol. 23, 1098-1104)

In the site-directed mutagenesis to obtain the Δd and Δe mutants of IgG1, the oligonucleotide encoding the Δd mutation (Q268) was MO29 (coding strand orientation):

SEQ ID NO: 26

5′ GTG GAC GTG AGC CAA GAA GAC CCT GAG 3′

The oligonucleotide encoding the Δe mutation (E268) was MO29BACK (complementary strand orientation):

SEQ ID NO: 27

5′ CTC AGG GTC TTC TTC GCT CAC GTC CAC 3′

The template for the first set of polymerase chain reactions was the IgG1 constant region in M13 (as described WO99/58572 (Cambridge University Technical Services)). MO29 was used in conjuction with the universal M13-40 primer to amplify from the mutation site to the 3′ end of the constant region. MO29BACK was used with MO10BACK to amplify from 5′ of the CH2 exon to the mutation site. Amplification was carried out over 15 cycles using Pfu DNA polymerase (Stratagene) and DNA products of the expected sizes were purified from an agarose gel using Prep-A-Gene matrix (BioRad). Overlap extension PCR with the universal M13-40 primer and MO10BACK was used to join these products in a reaction carried out over 15 cycles with Pfu DNA polymerase. Product of the expected length, containing the CH2 and CH3 exons, was gel purified, digested with XhoI and NotI and cloned to replace the similar fragment of the wildtype IgG1 vector, pSVgptFog1VHHuIgG1 (as described WO99/58572 (Cambridge University Technical Services)). The CH2 region of six of the resulting clones was nucleotide sequenced and all were found to be mutant, some encoding Q268 and some E268 as expected. For one G1Δd clone and one G1Δe clone, the DNA sequences of the entire CH2 and CH3 regions were determined to confirm that no spurious mutations had occurred during PCR and further sequencing confirmed that the Fog-1 VH and wildtype IgG1 CH1 and hinge regions were present.

To obtain the Δacd and Δace mutants of IgG1, the same procedure was carried out but using the G1Δac constant region DNA (as described WO99/58572) as template. Thus this method is easily adapted to provide other variants of the invention by using alternative template DNA. It is also simple to design an alternative version of oligonucleotide MO29 or MO29BACK such that the triplet corresponding to position 268 encodes a different amino acid, thereby providing variants with residues other than Q or E at position 268.

The heavy chain expression vectors for the Fog-1 G1Δd and Fog-1 G1Δe antibodies were each cotransfected with the kappa chain vector pSVhygFog1VKHuCK into the rat myeloma cell line YB2/0, antibody-secreting cells were expanded and antibodies purified essentially as described in UK Patent Application No: 9809951.8 (page 39 line 10—page 40 line 12).

The concentration of all relevant antibodies was checked in relation to the Fog-1 G1 antibody acting as standard. This was done in ELISAs which used either goat anti-human κ chain antibodies (Harlam) or anti-human IgG, Fc-specific antibodies (Sigma) as the capture reagent and HRPO-conjugated goat anti-human κ chain antibodies (Sigma) for detection. Reducing SDS-PAGE was used to confirm the integrity of the antibodies.

### Fluorescent Staining of FcγR Transfectants

Antibodies to be tested were combined with a equimolar amount of goat anti-human κ-chain F(ab′)₂ molecules (Rockland) in PBS containing 0.1% (w/v) NaN₃, 0.1% (w/v) BSA (wash buffer). Two-fold serial dilutions were made in wash buffer and incubated at 37 C for 2 h to allow complexes to form. The samples were cooled to 0 C before mixing with cells. The negative control test antibody was human IgA1, κ purified myeloma protein (The Binding Site) which should form complexes with the goat anti-κ F(ab′)₂ fragments but not contribute to binding by interacting with FcγRII itself.

Transfectants of the mouse 3T6 fibroblast cell line, which express FcγRIIa 131R or 131H cDNAs (Warmerdam et al., 1990 J. Exp. Med. 172:19-25) or FcγRIIb1* cDNA (Warmerdam et al., 1993 Int. Immunol. 5: 239-247), were obtained as single cell suspensions in wash buffer following treatment with cell dissociation buffer (Gibco BRL). Cells were pelleted at 10⁵ cells/well in 96-well plates, resuspended in 100 ml samples of complexed test antibody and incubated on ice for 30 min. Cells were washed three times with 150 ml/well wash buffer. The cells were incubated with a 1 in 100 dilution in wash buffer of FITC-conjugated rabbit F(ab′)₂ molecules specific for F(ab′)₂ fragments of goat IgG (Jackson). After washing, the cells were fixed in wash buffer containing 1% (v/v) formaldehyde. Fluorescence intensities of 20 000 events per sample were measured on a FACScan (Becton Dickinson) and the geometric mean obtained using LysisII software. The fluorescence is measured on an arbitrary scale and mean values cannot be compared between experiments carried out on different days. Surface expression of FcγRII was confirmed by staining with CD32 mAb AT10 (Serotec), followed by FITC-conjugated goat anti-mouse IgG Ab (Sigma). Fluorescence histograms showed a single peak suggesting uniform expression of FcγRII.

Transfectants expressing FcγRI cDNA, B2KA and 3T3+FcγRIa+γ-chain (van Urgt, M. J., Heijnen, I. A. F. M., Capel, P. J. A., Park, S. Y., Ra, C., Saito, T., Verbeek, J. S. and van de Winkel, J. G. J. (1996) FcR γ-chain is essential for both surface expression and function of human FcγRI (CD64) in vivo. Blood 87, 3593-3599), may be obtained as single cell suspensions in phosphate-buffered saline containing 0.1% (w/v) NaN₃, 0.1% (w/v) BSA (wash buffer) following treatment with cell dissociation buffer (Gibco BRL). Cells are pelleted at 10⁵ cells/well in 96-well plates, resuspended in 100 μl dilutions of the CAMPATH-1 or Fog-1 Ab and incubated on ice for 30 min. Cells are washed three times 150 μl/well wash buffer and similarly incubated with 20 μg/ml biotin-conjugated goat anti-human κ-chain Ab (Sigma) and then with 20 μg/ml ExtrAvidin-FITC (Sigma). After the final wash, cells are fixed in 100 μl wash buffer containing 1% (v/v) formaldehyde. Surface expression of FcγRI is confirmed by staining with CD64 mAb (Serotec) and FITC-conjugated goat and mouse IgG Ab (Sigma). Fluorescence intensities are measured on a FACScan (Becton Dickinson).

For transfectants bearing FcγRIIIb, CHO+FcγRIIIb NA1 or NA2 (Bux, J., Kissel, K., Hofmann, C. and Santoso, S. (1999) The use of allele-specific recombinant Fc gamma receptor IIIb antigens for the detection of granulocyte antibodies. Blood 93, 357-362), staining is carried out as described for 3T6+FcγRIIa 131H/H cells above.

An ability to trigger complement dependent lysis (which will generally be through an increased affinity for the C1q molecule) can be measured by CR-51 release from target cells in the presence of the complement components e.g. in the form of serum. Similarly, cell mediated destruction of the target may be assessed by CR-51 release from target cells in the presence of suitable cytotoxic cells e.g. blood mononuclear effector cells (as described WO99/58572 (Cambridge University Technical Services).

### Discussion

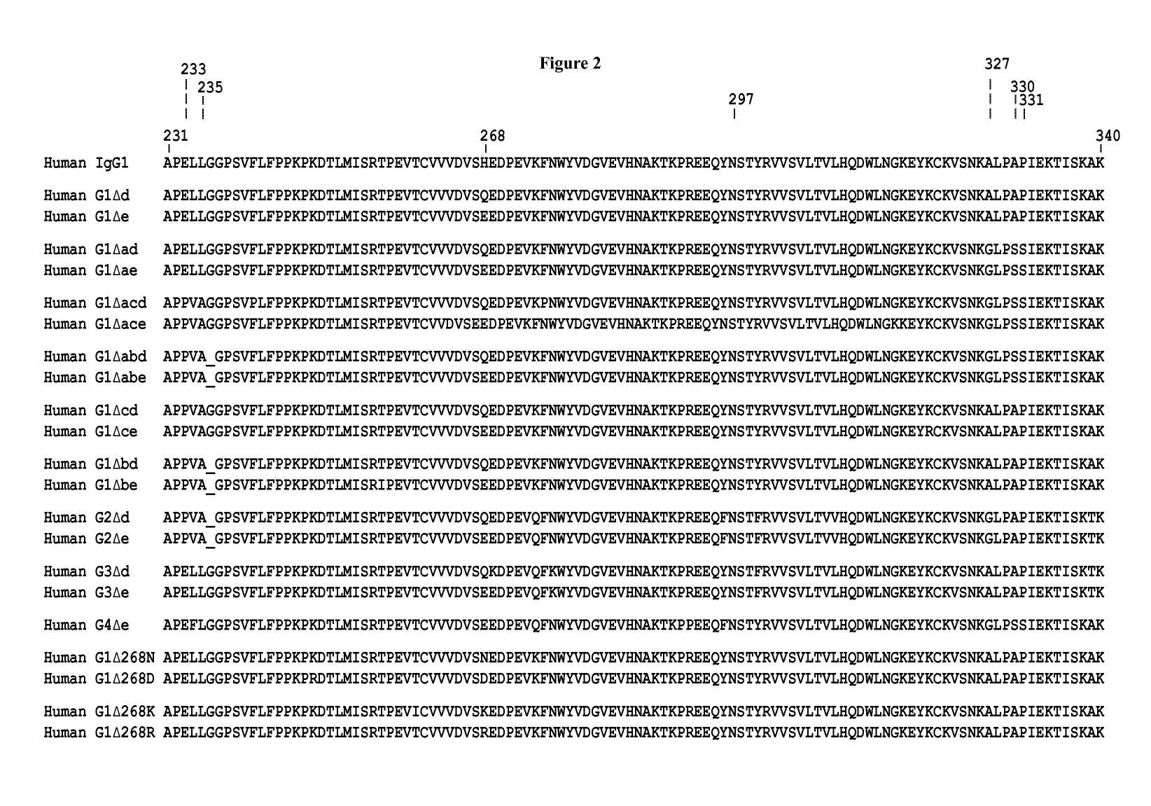

As shown in FIG. 2, the G1Δd constant region is an example of a native IgG1 constant region with the substitution of a polar amino acid (Gln) at position 268. Thus, the variant CH2 region is identical to the native IgG1 CH2 region except at position 268. The G1Δe constant region is an example of a native IgG1 constant region with the substitution of a negatively-charged amino acid (Glu) at position 268. Again, the variant CH2 region is identical to the native IgG1 CH2 region except at position 268. In the mutants G1Δacd and G1Δace, the substitutions at position 268 are made on a CH2 region which carries six residue changes compared with the native IgG1 CH2 region.

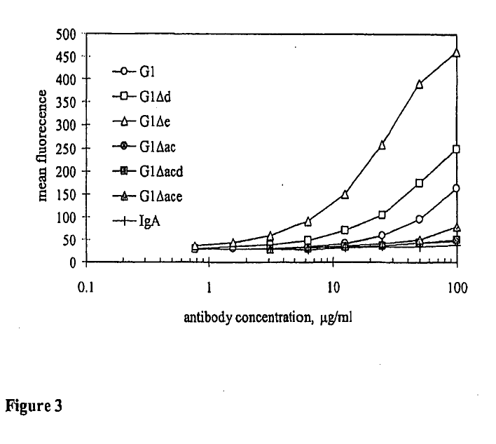

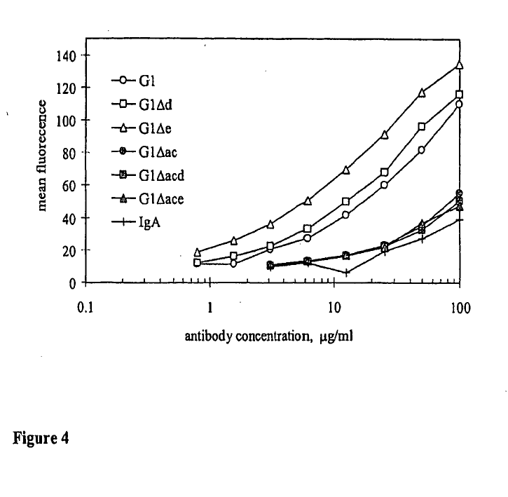

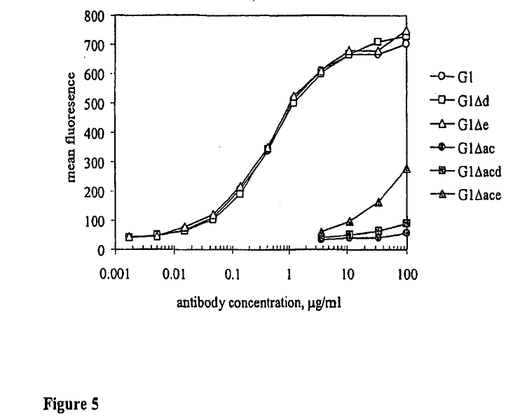

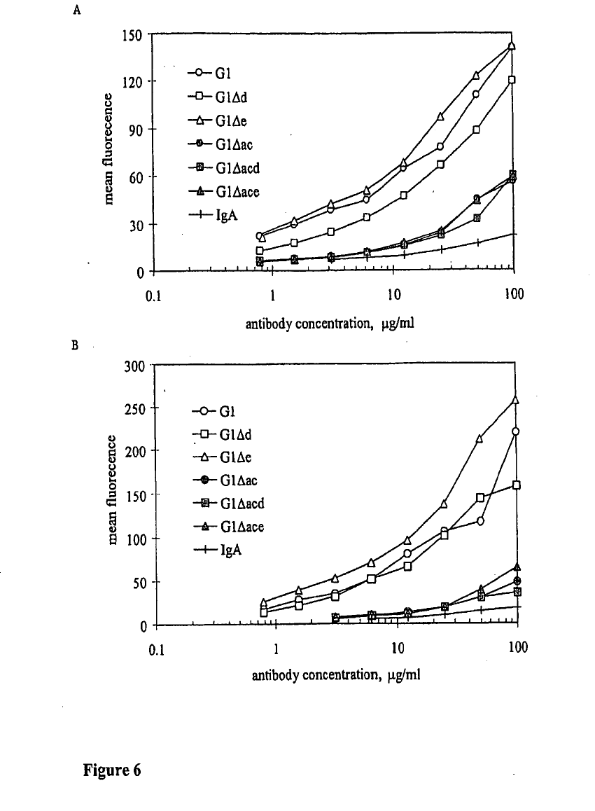

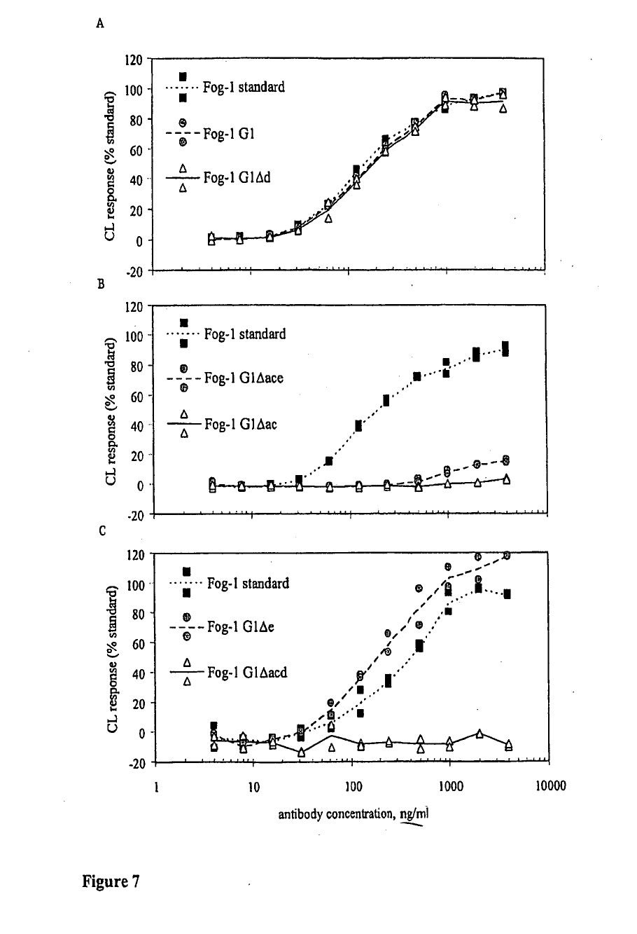

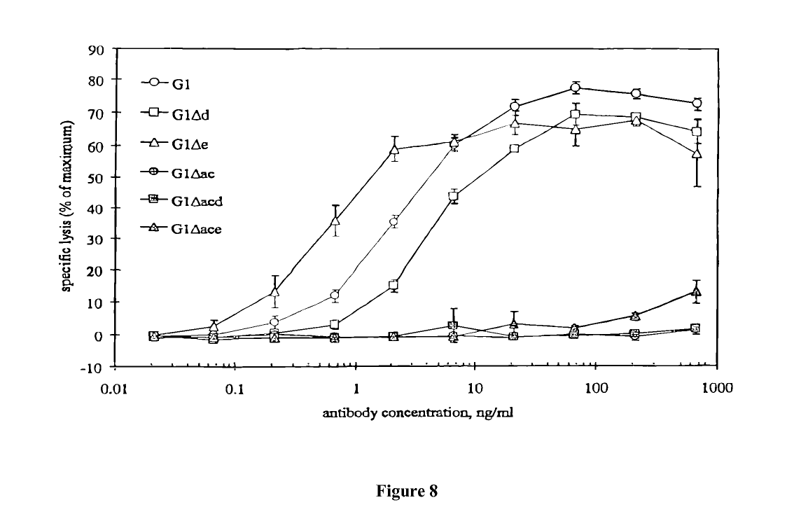

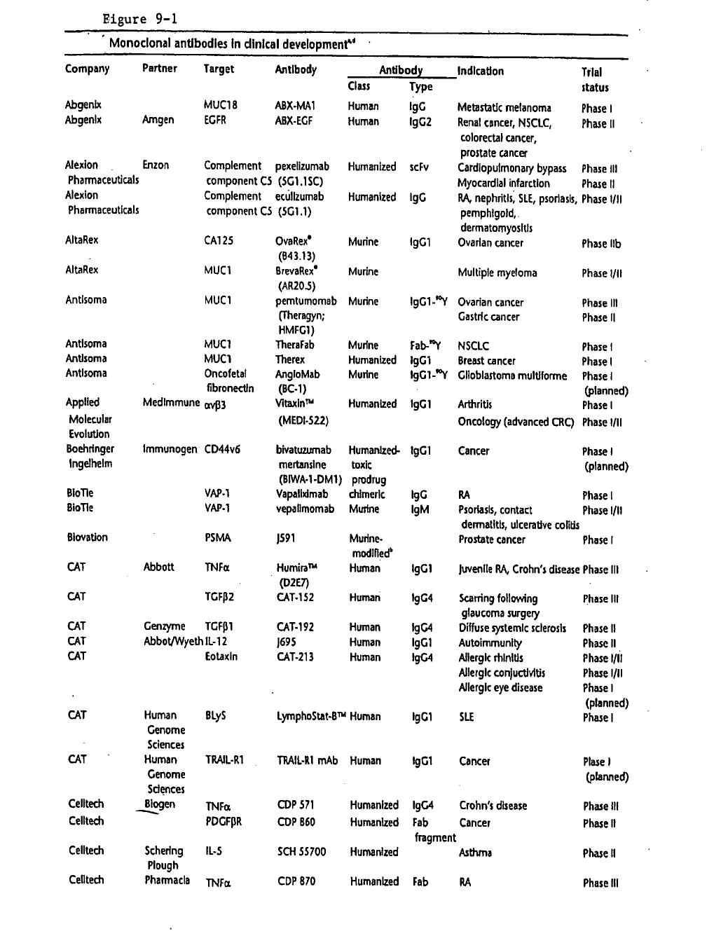

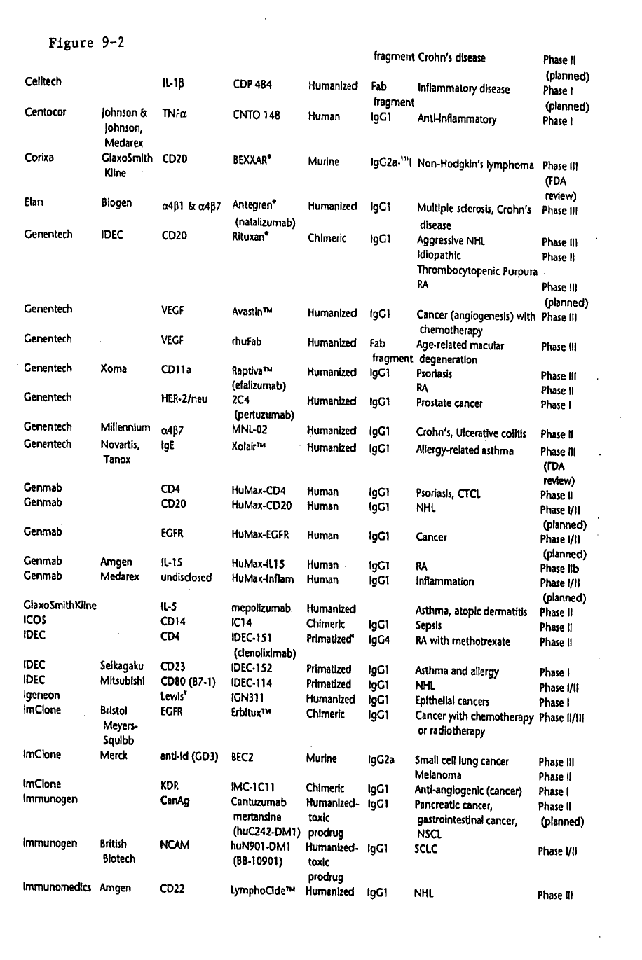

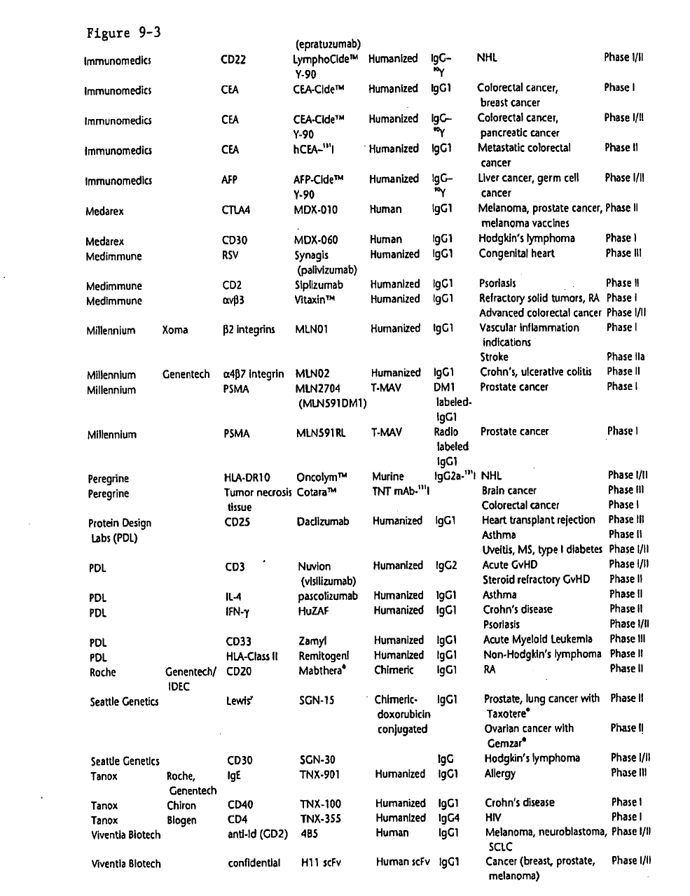

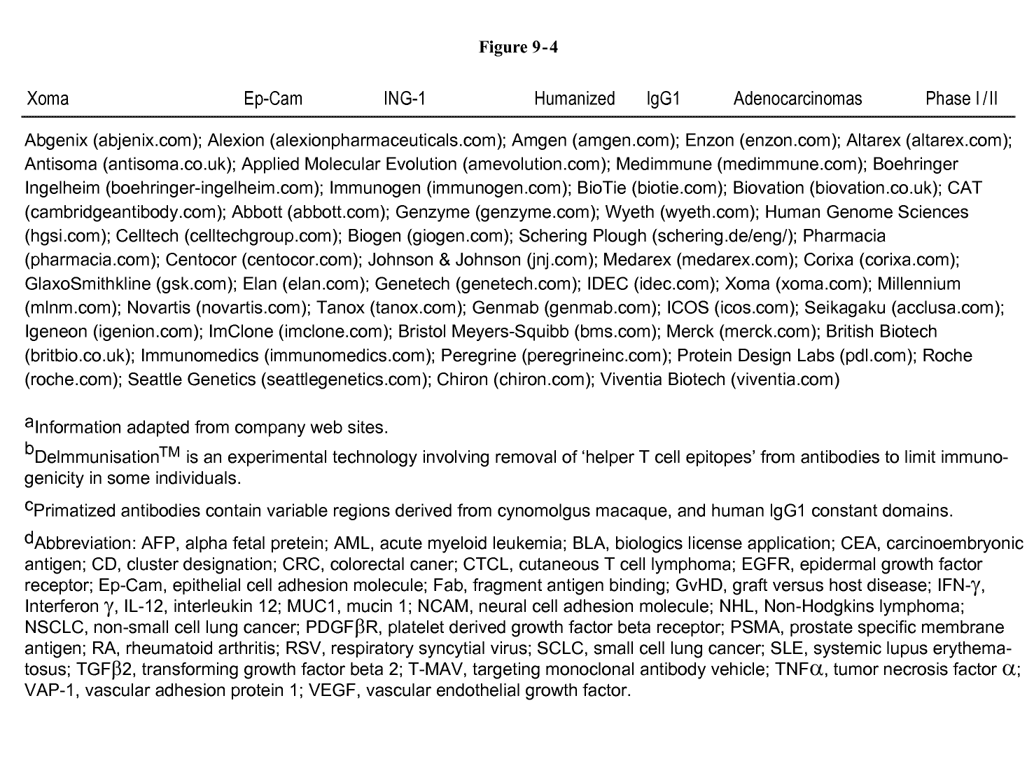

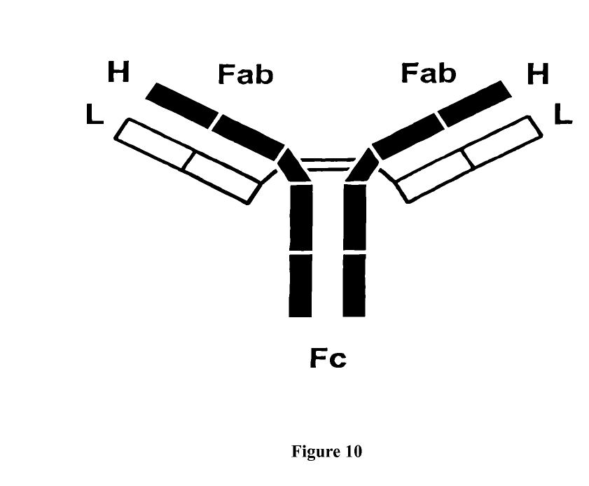

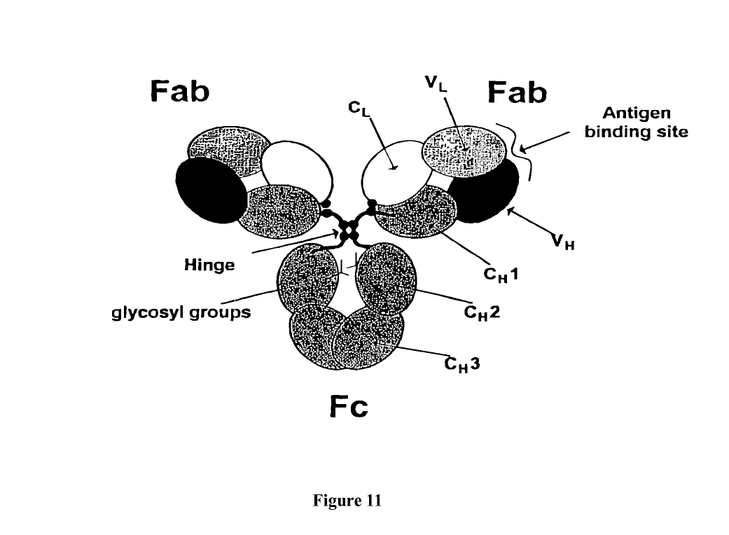

FIGS. 3 to 8 illustrate the functions of some example embodiments of the invention. Notably, G1Δd exhibits a small increase (two-fold) in binding to FcγRIIb relative to the native IgG1. G1Δe is two-fold more active than G1 in FcγRIIa 131H binding, monocyte chemiluminescence, FcγRIIIb and ADCC but eight-fold more active in FcγRIIb binding (enhanced ADCC is good evidence for increased binding activity with the FcγRIIIa (CD16) receptor as expressed on NK-cells). Thus G1Δe mediates enhanced cellular cytotoxicity and enhanced effector cell activation when compared to native IgG1. For G1Δe and G1Δd an increase in relative binding affinity for FcγRIIb compared to FcγRIIa has been demonstrated. Effects of the Δe mutation are also seen on the G1Δac background (G1Δace). In assays of FcγRI binding, monocyte chemiluminescence and ADCC, G1Δace shows activity at high concentration when the corresponding activity of G1Δac is at background levels.

### REFERENCES

Dyer M J S, Hale G, Marcus R, Waldmann H (1990) Remission induction in patients with lymphoid malignancies using unconjugated CAMPATH-1 monoclonal antibodies. Leukaemia and Lymphoma, 2: 179-.
Glennie, M J, Johnson W M (2000) Clinical trials of antibody therapy. Immunology Today 21: 403-410
Glennie, M J, van de Winkel, J G J (2003) Renaissance of cancer therapeutic antibodies. Drug Discovery Today 8: 503-510
Hale G, Bright S, Chumbley G, Hoang T, Metcalf D, Munro A J, Waldmann H (1983) Removal of T cells from bone marrow for transplantation: amonoclonal antilymphocyte antibody that fixes human complement. Blood, 62: 873-882.
Hale G, Waldmann H (1996) Recent results using CAMPATH-1 antibodies to control GVHD and graft rejection. Bone Marrow Transplant, 17: 305-308.
Hale G, Dyer M J S, Clark M R, Phillips J M, Marcus R, Riechmann L,
Winter G, Waldmann H (1988) Remission induction in non-Hodgkin lymphoma with reshaped human monoclonal antibody CAMPATH-1H. Lancet, 2: 1394-1399.
Kumpell, B M (2002) On the mechanism of tolerance to the Rh D antigen mediated by passive anti-D (Rh-D prophylaxis) Immunology Letters 82: 67-73
Lockwood C M, Thiru S, Isaacs J D, Hale G, Waldmann H (1993) Long-term remission of intractable systemic vasculitis with monoclonal antibody therapy. Lancet, 341: 1620-1622.
Mathieson P W, Cobbold S P, Hale G, Clark M R, Oliveira D B G, Lockwood C M, Wladmann H (1990) Monoclonal antibody therapy in systemic vasculitis. New Engl J Med, 323: 250-254.
Matteson E L, Yocum D E, St-Clair E W, Achkar A A, Thakor M S, Jacobs M R,
Hays A E, Heitman-C K, Johnston J M (1995) Treatment of active refractory rheumatoid arthritis with humanized monoclonal antibody CAMPATH-1H administered by daily subcutaneous injection. Arthritis Rheum, 38: 1187-1193.
Moreau T, Thorpe J, Miller D, Moseley I, Hale G, Waldmann H, Clayton D, Wing M, Scolding N, Compston A (1994) Preliminary evidence from magnetic resonance imaging for reduction in disease activity after lymphocyte depletion in multiple sclerosis. Lancet, 344: 298-301.
Riechmann L, Clark M R, Waldmann H, Winter G (1988) Reshaping human antibodies for therapy. Nature, 332: 323-327.
Xia M Q, Tone M, Packman L, Hale G, Waldmann H (1991) Characterization of the CAMPATH-1 (CDw52) antigen: biochemical analysis and cDNA cloning reveal an unusually small peptide backbone. Eur J Immunol, 21: 1677-1684.

1. An isolated IgG antibody comprising a CH2 region, wherein said CH2 region is human but for Asp at position 268 according to the EU numbering system.

2. An isolated IgG antibody comprising a CH2 region which contains Asp at position 268 according to the EU numbering system.

3. An isolated human IgG antibody comprising Asp at position 268 according to the EU numbering system.

4. The antibody of claim 1 wherein said antibody is a human antibody but for said Asp at position 268 according to the EU numbering system.

5. The antibody of claim 2 wherein said antibody is a human antibody, but for said Asp at position 268 according to the EU numbering system.

6. The antibody of claim 1 wherein said antibody has greater FcγRIII binding activity as compared to the same antibody where said amino acid at said position 268 is His or Gln.

7. The antibody of claim 2 or claim 3 wherein said antibody has greater FcγRIII binding activity as compared to the same antibody where said amino acid at said position 268 is His or Gln.

8. The antibody of claim 2 further comprising an Asn at amino acid position 297 according to the EU numbering system.

9. The antibody of claim 1 further comprising an Asn at amino acid position 297 according to the EU numbering system.

10. The antibody of claim 8 or claim 9 wherein said Asn is glycosylated.

11. The antibody of claim 1 or claim 2 or claim 3 wherein said antibody binds a target selected from the group consisting of MUC18, EGFR, Complement component C5, CA125, MUC1, Oncofetal fibronectin, αvβ3, CD44v6, VAP-1, PSMA, TNFα, TGFβ2, TGFβ1, IL-12, Eotaxin, BLyS, TRAIL-R1, PDGFβR, IL-5, IL-1β, CD20, α4β1, VEGF, CD11a, HER-2/neu, α4β7, IgE, EGFR, IL-15, IL-5, CD14, CD4, CD23, CD80, Lewis^{y}, anti-Id (GD3), KDR, CanAg, NCAM, CD22, CEA, AFP, CTLA4, CD30, RSV, CD2, β2 integrin, α4β7 integrin, PSMA, HLA-DR10, Tumor necrosis tissue, CD25, CD3, IL-4, IFN-γ, CD33, HLA-Class II, CD30, CD40, and anti-Id (GD2).

12. The antibody of claim 1 or claim 2 or claim 3 wherein said antibody is a humanized antibody.

13. The antibody of claim 1 or claim 2 or claim 3, wherein the amino acid sequence of said antibody is the same, except for the amino acid of said position 268, as the amino acid sequence of an antibody selected from the group consisting of ABX-MA1, ABX-EGF, pexelizumab (5G1.1SC), eculizumab (5G1.1), B43.13, ARZ0.5, pemtumomab, THERAGYN HMFG1 (Pemtumomab), Therafab (radiolabelled monoclonal antibody fragment that binds to MUC 1 antigen), Angiomab (muBC-1 murine antibody radiolabelled), MEDI-522, bivatuzumab mertansine (BIWA-1-DM1), Vapaliximab, vepalimomab, J591, D2E7 (adalimumab), CAT-152, CAT-192, J695, CAT-213, LYMPHOSTAT-B (belimumab), TRAIL-R1, CDP 571, CDP 860, SCH 55700, CDP 870, CDP 484, CNTO 148, BEXXAR (Tositumomab and Iodine I 131 Tositumomab), natalizumab, RITUXAN (rituximab), AVASTIN (bevacizumab), RHUFAB (ranibizumab), efalizumab, 2C4, MNL-02, XOLAIR (omalizumab), HuMax-CD4, HuMax-CD20, zalutumumab, HuMax-IL15, HuMax-Inflam, mepolizumab, IC14, IDEC-151, IDEC-152, IDEC-114, IGN311, ERBITUX (cetuximab), BEC2, IMC-1C11, Cantuzumab mertansine, huN901-DM1, LYMPHOCIDE (epratuzumab), LYMPHOCIDE Y-90 epratuzumab and Yttrium Y 90), CEA-CIDE (labetuzumab), CEA-CIDE Y-90 (labetuzumab and Yttrium Y 90), hCEA-¹³¹l (humanized CEA antibody labeled with Iodine 131), AFP-Cide Y-90 (yttrium y 90 tacatuzumab), MDX-010, MDX-060, palivizumab, Siplizumab, VITAXIN (an anti alphavbeta3 antibody), MLN01, MLN02, MLN2704, MLN591RL, ONCOLYM (valacyclovir—131I Lym 1), Daclizumab, visilizumab, pascolizumab, HuZAF (fontolizumab an anti-Interferon-gamma monoclonal antibody), ZAMYL (Smart M195), Remitogen (Hu1D10 anti-HLA-DR), MABTHERA (rituximab), SGN-15, SGN-30 (anti-CD30 antibody), TNX-901, TNX-100, TNX-355, 4BS, and H11 scFv.

14. The antibody of claim 1, wherein said antibody has increased relative binding for FcγRIIb as compared to binding to FcγRIIa, as compared to the same antibody where said amino acid at said position 268 is His or Gln.

15. The antibody of claim 2 or claim 3, wherein said antibody has increased relative binding for FcγRIIb as compared to binding to FcγRIIa, as compared to the same antibody where said amino acid at said position 268 is His or Gln.

16. A fusion protein comprising a binding molecule, said binding molecule comprising a binding domain capable of binding a target molecule and an effector domain comprising a variant CH2 polypeptide in which the amino acid at position 268 of the variant polypeptide is D (Asp).

17. A pharmaceutical preparation which comprises an antibody of claim 11, and a pharmaceutically acceptable carrier or diluent

18. A pharmaceutical preparation which comprises an antibody of claim 13, and a pharmaceutically acceptable carrier or diluent.

19. An isolated IgG antibody comprising a CH2 region, wherein said CH2 region is human but for Asp at position 268 according to the EU numbering system, said isolated IgG antibody having increased binding affinity for FcγRIII as compared to the same IgG antibody which does not contain Asp at position 268.

20. An isolated IgG antibody comprising a CH2 region which contains Asp at position 268 according to the EU numbering system, said isolated IgG antibody having increased binding affinity for FcγRIII as compared to the same IgG antibody which does not contain Asp at position 268.

21. An isolated human IgG antibody comprising Asp at position 268 according to the EU numbering system, said isolated human IgG antibody having increased binding affinity for FcγRIII as compared to the same IgG antibody which does not contain Asp at position 268.

22. The antibody of claim 19 wherein said antibody is a human antibody but for said Asp at position 268 according to the EU numbering system.

23. The antibody of claim 20 wherein said antibody is a human antibody, but for said Asp at position 268 according to the EU numbering system.

24. The antibody of claim 19 wherein said antibody has greater FcγRIII binding activity as compared to the same antibody where said amino acid at said position 268 is His or Gln.

25. The antibody of claim 20 or claim 21 wherein said antibody has greater FcγRIII binding activity as compared to the same antibody where said amino acid at said position 268 is His or Gln.

26. The antibody of claim 20 further comprising an Asn at amino acid position 297 according to the EU numbering system.

27. The antibody of claim 19 wherein the Asn at amino acid position 297 according to the EU numbering system is glycosylated.

28. The antibody of claim 26 wherein said Asn is glycosylated.

29. The antibody of claim 19 or claim 20 or claim 21 wherein said antibody binds a target selected from the group consisting of MUC18, EGFR, Complement component C5, CA125, MUC1, Oncofetal fibronectin, αvβ3, CD44v6, VAP-1, PSMA, TNFα, TGFβ2, TGFβ1, IL-12, Eotaxin, BLyS, TRAIL-R1, PDGFβR, IL-5, IL-1β, CD20, α4β1, VEGF, CD11a, HER-2/neu, α4β7, IgE, EGFR, IL-15, IL-5, CD14, CD4, CD23, CD80, Lewis^{y}, anti-Id (GD3), KDR, CanAg, NCAM, CD22, CEA, AFP, CTLA4, CD30, RSV, CD2, β2 integrin, α4β7 integrin, PSMA, HLA-DR10, Tumor necrosis tissue, CD25, CD3, IL-4, IFN-γ, CD33, HLA-Class II, CD30, CD40, and anti-Id (GD2).

30. The antibody of claim 19 or claim 20 or claim 21 wherein said antibody is a humanized antibody.

31. The antibody of claim 19 or claim 20 or claim 21, wherein the amino acid sequence of said antibody is the same, except for the amino acid of said position 268, as the amino acid sequence of an antibody selected from the group consisting of ABX-MA1, ABX-EGF, pexelizumab (5G1.1SC), eculizumab (5G1.1), B43.13, ARZ0.5, pemtumomab, THERAGYN HMFG1 (Pemtumomab), Therafab (radiolabelled monoclonal antibody fragment that binds to MUC 1 antigen), Angiomab (muBC-1 murine antibody radiolabelled), MEDI-522, bivatuzumab mertansine (BIWA-1-DM1), Vapaliximab, vepalimomab, J591, D2E7 (adalimumab), CAT-152, CAT-192, J695, CAT-213, LYMPHOSTAT-B (belimumab), TRAIL-R1, CDP 571, CDP 860, SCH 55700, CDP 870, CDP 484, CNTO 148, BEXXAR (Tositumomab and Iodine I 131 Tositumomab), natalizumab, RITUXAN (rituximab), AVASTIN (bevacizumab), RHUFAB (ranibizumab), efalizumab, 2C4, MNL-02, XOLAIR (omalizumab), HuMax-CD4, HuMax-CD20, zalutumumab, HuMax-IL15, HuMax-Inflam, mepolizumab, IC14, IDEC-151, IDEC-152, IDEC-114, IGN311, ERBITUX (cetuximab), BEC2, IMC-1C11, Cantuzumab mertansine, huN901-DM1, LYMPHOCIDE (epratuzumab), LYMPHOCIDE Y-90 epratuzumab and Yttrium Y 90), CEA-CIDE (labetuzumab), CEA-CIDE Y-90 (labetuzumab and Yttrium Y 90), hCEA-¹³¹l (humanized CEA antibody labeled with Iodine 131), AFP-Cide Y-90 (yttrium y 90 tacatuzumab), MDX-010, MDX-060, palivizumab, Siplizumab, VITAXIN (an anti alphavbeta3 antibody), MLN01, MLN02, MLN2704, MLN591RL, ONCOLYM (valacyclovir—131I Lym 1), Daclizumab, visilizumab, pascolizumab, HuZAF (fontolizumab an anti-Interferon-gamma monoclonal antibody), ZAMYL (Smart M195), Remitogen (Hu1D10 anti-HLA-DR), MABTHERA (rituximab), SGN-15, SGN-30 (anti-CD30 antibody), TNX-901, TNX-100, TNX-355, 4BS, and H11 scFv.

32. The antibody of claim 19, wherein said antibody has increased relative binding for FcγRIIb as compared to binding to FcγRIIa, as compared to the same antibody where said amino acid at said position 268 is His or Gln.

33. The antibody of claim 20 or claim 21, wherein said antibody has increased relative binding for FcγRIIb as compared to binding to FcγRIIa, as compared to the same antibody where said amino acid at said position 268 is His or Gln.


SEQUENCE LISTING (27 sequences)

SEQ ID NO: 1 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 2 - protein, 109 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 3 - protein, 110 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 4 - protein, 110 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 5 - protein, 110 aa, Artificial Sequence
  Variant of Human IgG1 CH2 sequence with delta d (Q268) mutation
APELLGGPSVFLFPPKPKDTLMISRTPEVTCVVVDVSQEDPEVKFNWYVDGVEVHNAKTKPREEQYNSTYRVVSVLTVLHQDWLNGKEYKCKVSNKALPAPIEKTISKAK

SEQ ID NO: 6 - protein, 110 aa, Artificial Sequence
  Variant of Human IgG1 CH2 sequence with delta e (E268) mutation
APELLGGPSVFLFPPKPKDTLMISRTPEVTCVVVDVSEEDPEVKFNWYVDGVEVHNAKTKPREEQYNSTYRVVSVLTVLHQDWLNGKEYKCKVSNKALPAPIEKTISKAK

SEQ ID NO: 7 - protein, 110 aa, Artificial Sequence
  Variant of Human IgG1 CH2 sequence with delta a and d (Q268) mutations
APELLGGPSVFLFPPKPKDTLMISRTPEVTCVVVDVSQEDPEVKFNWYVDGVEVHNAKTKPREEQYNSTYRVVSVLTVLHQDWLNGKEYKCKVSNKGLPSSIEKTISKAK

SEQ ID NO: 8 - protein, 110 aa, Artificial Sequence
  Variant of Human IgG1 CH2 sequence with delta a and e (E268) mutations
APELLGGPSVFLFPPKPKDTLMISRTPEVTCVVVDVSEEDPEVKFNWYVDGVEVHNAKTKPREEQYNSTYRVVSVLTVLHQDWLNGKEYKCKVSNKGLPSSIEKTISKAK

SEQ ID NO: 9 - protein, 110 aa, Artificial Sequence
  Variant of Human IgG1 CH2 sequence with delta a, c and d (D268) mutations
APPVAGGPSVFLFPPKPKDTLMISRTPEVTCVVVDVSQEDPEVKFNWYVDGVEVHNAKTKPREEQYNSTYRVVSVLTVLHQDWLNGKEYKCKVSNKGLPSSIEKTISKAK

SEQ ID NO: 10 - protein, 110 aa, Artificial Sequence
  Variant of Human IgG1 CH2 sequence with delta a, c and e (E268) mutations
APPVAGGPSVFLFPPKPKDTLMISRTPEVTCVVVDVSEEDPEVKFNWYVDGVEVHNAKTKPREEQYNSTYRVVSVLTVLHQDWLNGKEYKCKVSNKGLPSSIEKTISKAK

SEQ ID NO: 11 - protein, 109 aa, Artificial Sequence
  Variant of Human IgG1 CH2 sequence with delta a, b and d (D268) mutations
APPVAGPSVFLFPPKPKDTLMISRTPEVTCVVVDVSQEDPEVKFNWYVDGVEVHNAKTKPREEQYNSTYRVVSVLTVLHQDWLNGKEYKCKVSNKGLPSSIEKTISKAK

SEQ ID NO: 12 - protein, 109 aa, Artificial Sequence
  Variant of Human IgG1 CH2 sequence with delta a, b and e (E268) mutations
APPVAGPSVFLFPPKPKDTLMISRTPEVTCVVVDVSEEDPEVKFNWYVDGVEVHNAKTKPREEQYNSTYRVVSVLTVLHQDWLNGKEYKCKVSNKGLPSSIEKTISKAK

SEQ ID NO: 13 - protein, 110 aa, Artificial Sequence
  Variant of Human IgG1 CH2 sequence with delta c and d (D268) mutations
APPVAGGPSVFLFPPKPKDTLMISRTPEVTCVVVDVSQEDPEVKFNWYVDGVEVHNAKTKPREEQYNSTYRVVSVLTVLHQDWLNGKEYKCKVSNKALPAPIEKTISKAK

SEQ ID NO: 14 - protein, 110 aa, Artificial Sequence
  Variant of Human IgG1 CH2 sequence with delta c and e (E268) mutations
APPVAGGPSVFLFPPKPKDTLMISRTPEVTCVVVDVSEEDPEVKFNWYVDGVEVHNAKTKPREEQYNSTYRVVSVLTVLHQDWLNGKEYKCKVSNKALPAPIEKTISKAK

SEQ ID NO: 15 - protein, 109 aa, Artificial Sequence
  Variant of Human IgG1 CH2 sequence with delta b and d (D268) mutations
APPVAGPSVFLFPPKPKDTLMISRTPEVTCVVVDVSQEDPEVKFNWYVDGVEVHNAKTKPREEQYNSTYRVVSVLTVLHQDWLNGKEYKCKVSNKALPAPIEKTISKAK

SEQ ID NO: 16 - protein, 109 aa, Artificial Sequence
  Variant of Human IgG1 CH2 sequence with delta b and e (E268) mutations
APPVAGPSVFLFPPKPKDTLMISRTPEVTCVVVDVSEEDPEVKFNWYVDGVEVHNAKTKPREEQYNSTYRVVSVLTVLHQDWLNGKEYKCKVSNKALPAPIEKTISKAK

SEQ ID NO: 17 - protein, 109 aa, Artificial Sequence
  Variant of Human IgG2 CH2 sequence with delta d (D268) mutation
APPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTK

SEQ ID NO: 18 - protein, 109 aa, Artificial Sequence
  Variant of Human IgG2 CH2 sequence with delta e (E268) mutation
APPVAGPSVFLFPPKPKDTLMISRTPEVTCVVVDVSEEDPEVQFNWYVDGVEVHNAKTKPREEQFNSTFRVVSVLTVVHQDWLNGKEYKCKVSNKGLPAPIEKTISKTK

SEQ ID NO: 19 - protein, 110 aa, Artificial Sequence
  Variant of Human IgG3 CH2 sequence with delta d (D268) mutation
APELLGGPSVFLFPPKPKDTLMISRTPEVTCVVVDVSQEDPEVQFKWYVDGVEVHNAKTKPREEQYNSTFRVVSVLTVLHQDWLNGKEYKCKVSNKALPAPIEKTISKTK

SEQ ID NO: 20 - protein, 110 aa, Artificial Sequence
  Variant of Human IgG3 CH2 sequence with delta e (E268) mutation
APELLGGPSVFLFPPKPKDTLMISRTPEVTCVVVDVSEEDPEVQFKWYVDGVEVHNAKTKPREEQYNSTFRVVSVLTVLHQDWLNGKEYKCKVSNKALPAPIEKTISKTK

SEQ ID NO: 21 - protein, 110 aa, Artificial Sequence
  Variant of Human IgG4 CH2 sequence with delta e (E268) mutation
APEFLGGPSVFLFPPKPKDTLMISRTPEVTCVVVDVSEEDPEVQFNWYVDGVEVHNAKTKPREEQFNSTYRVVSVLTVLHQDWLNGKEYKCKVSNKGLPSSIEKTISKAK

SEQ ID NO: 22 - protein, 110 aa, Artificial Sequence
  Variant of Human IgG1 CH2 sequence with delta 268N mutation
APELLGGPSVFLFPPKPKDTLMISRTPEVTCVVVDVSNEDPEVKFNWYVDGVEVHNAKTKPREEQYNSTYRVVSVLTVLHQDWLNGKEYKCKVSNKALPAPIEKTISKAK

SEQ ID NO: 23 - protein, 110 aa, Artificial Sequence
  Variant of Human IgG1 CH2 sequence with delta 268D mutation
APELLGGPSVFLFPPKPKDTLMISRTPEVTCVVVDVSDEDPEVKFNWYVDGVEVHNAKTKPREEQYNSTYRVVSVLTVLHQDWLNGKEYKCKVSNKALPAPIEKTISKAK

SEQ ID NO: 24 - protein, 110 aa, Artificial Sequence
  Variant of Human IgG1 CH2 sequence with delta 268K mutation
APELLGGPSVFLFPPKPKDTLMISRTPEVTCVVVDVSKEDPEVKFNWYVDGVEVHNAKTKPREEQYNSTYRVVSVLTVLHQDWLNGKEYKCKVSNKALPAPIEKTISKAK

SEQ ID NO: 25 - protein, 110 aa, Artificial Sequence
  Variant of Human IgG1 CH2 sequence with delta 268R mutation
APELLGGPSVFLFPPKPKDTLMISRTPEVTCVVVDVSREDPEVKFNWYVDGVEVHNAKTKPREEQYNSTYRVVSVLTVLHQDWLNGKEYKCKVSNKALPAPIEKTISKAK

SEQ ID NO: 26 - DNA, 27 bp, Artificial Sequence
  M029 oligonucleotide for delta d mutation (Q268)
gtggacgtgagccaagaagaccctgag

SEQ ID NO: 27 - DNA, 27 bp, Artificial Sequence
  M029BACK oligonucleotide for delta e mutation (E268)
ctcagggtcttcttcgctcacgtccac